(19) United States
(12) Patent Application Publication
(10) Pub. No.: US 2012/0101322 A1
(43) Pub. Date: Apr. 26, 2012

(54) OXYGENATE CONVERSION CATALYST, PROCESS FOR THE PREPARATION OF AN OLEFINIC PRODUCT, AND PROCESS FOR THE PREPARATION OF AN OXYGENATE CONVERSION CATALYST

(76) Inventors: Leslie Andrew Chewter, Amsterdam (NL); Jeroen Van Westrenen, Amsterdam (NL); Ferry Winter, Amsterdam (NL)

(21) Appl. No.: 13/320,958

(22) PCT Filed: May 18, 2010
(86) PCT No.: PCT/EP2010/056833

§ 371 Date: Dec. 20, 2011

(30) Foreign Application Priority Data
May 19, 2009 (EP) 09160617.8

(51) Int. Cl. C07C 1/20; B01J 29/80; B01J 29/70

(52) U.S. Cl. 585/638; 502/60; 502/67

(57) ABSTRACT

An oxygenate conversion catalyst comprising both a first molecular sieve having one-dimensional 10-membered ring channels, and a second molecular sieve having more-dimensional channels, wherein the second molecular sieve comprises MEL-type aluminosilicate; a process for the preparation of an olefinic product in the presence of the oxygenate conversion catalyst, and a process for the preparation of an oxygenate conversion catalyst.

This invention relates to an oxygenate conversion catalyst, a process for the preparation of an olefinic product, and to a process for the preparation of an oxygenate conversion catalyst. The invention is useful for the preparation of an olefin or olefinic product, especially lower olefins such as ethylene and/or propylene. In particular this invention relates to the conversion of an oxygenate feedstock into olefins.

Processes for the preparation of olefins from oxygenates are known in the art. Of particular interest is often the production of light olefins, in particular ethylene and/or propylene. The oxygenate feedstock can for example comprise methanol and/or dimethylether, and an interesting route includes their production from synthesis gas derived from e.g. natural gas or via coal gasification.

For example, WO2007/135052 discloses a process wherein an alcohol and/or ether containing oxygenate feedstock and an olefinic co-feed are reacted in the presence of a zeolite having one-dimensional 10-membered ring channels to prepare an olefinic reaction mixture, and wherein part of the obtained olefinic reaction mixture is recycled as olefinic co-feed. With a methanol and/or dimethylether containing feedstock, and an olefinic co-feed comprising C4 and/or C5 olefins, an olefinic product rich in light olefins can be obtained.

WO2004/056944 discloses a process for cracking C4-C8 hydrocarbons, to prepare olefins, especially propylene, wherein a catalyst with a combination of ZSM-12 with either ZSM-5 or ZSM-23 is used. No reference to oxygenates is made.

U.S. Pat. No. 6,797,851 describes a process for making ethylene and propylene from an oxygenate feed. The process is conducted in two stages using two different zeolite catalysts, wherein in the first stage oxygenates are converted to a light olefin stream, and wherein in the second stage C4+ olefins produced in the first stage are converted to additional ethylene and propylene. The only zeolite that is disclosed for the first step is ZSM-5. For the second stage, zeolite ZSM-22 and ZSM-35 are disclosed in experiments. Various embodiments of reaction systems with first and second stage catalyst in separate reaction zones are discussed. Without disclosing an embodiment, it is generally mentioned that the two catalysts can be mixed.

It is desired to provide a process and suitable catalyst to maximise production of light olefins, and in a particular aspect maximise the production of ethylene, from an oxygenate feedstock.

According to a first aspect of the present invention, there is provided an oxygenate conversion catalyst comprising both a first molecular sieve having one-dimensional 10-membered ring channels, and a second molecular sieve having more-dimensional channels, wherein the second molecular sieve comprises MEL-type aluminosilicate. In a particular embodiment, the oxygenate conversion catalyst comprises particles and individual catalyst particles comprise both the first molecular sieve and the second molecular sieve, in particular MEL-type aluminosilicate. Typically the molecular sieves comprise or consist of crystals. When referring to a second molecular sieve hereinafter, it shall always comprise MEL-type aluminosilicate, unless explicitly stated otherwise.

According to a second aspect of the present invention, there is provided a process for the preparation of an olefinic product, which process comprises reacting an oxygenate feedstock in a reaction zone in the presence of the oxygenate conversion catalyst according to the first aspect of the invention.

When the oxygenate conversion catalyst comprises particles and individual catalyst particles comprise both the first molecular sieve and the second molecular sieve, the first and second molecular sieves are intimately mixed. I.e., crystals of the first and second molecular sieves are present in the same particle.

Preferably therefore an average distance between a crystal of the first molecular sieve and a crystal of the second molecular sieve is less than an average particle size of the catalyst particles, preferably 40 μm or less, more preferably 20 μm or less, especially 10 μm or less. For near-spherical particles the average particle size can be determined by the weight-averaged diameter of a statistically representative quantity of particles, such as of e.g. 10 mg, 100 mg, 250 mg, or 1 g of particles. Such a statistically representative quantity of particles is referred to herein as a bed of particles. For other shapes of catalyst particles the skilled person knows how to define a suitable average of a characteristic dimension as average particle size, preferably a weight-average is used. The average distance between a crystal of the first molecular sieve and a crystal of the second molecular sieve can be determined using for instance electron-microscopy.

It is however also possible that the oxygenate conversion catalyst comprises a mixture of catalyst particles where individual particles include one or the other of the molecular sieve types, not both. The catalyst can also be a mixture of molecular sieves as such.

Preferably the oxygenate conversion catalyst, such as a bed of the catalyst particles, comprises at least 1 wt % and less than 50 wt % of the second molecular sieve, based on the total weight of first and second molecular sieves in the bed; preferably at least 5 wt % and less than 40 wt %, more preferably at least 8 wt % and less than 25 wt %. For certain embodiments the second molecular sieve may be present at less than 18 wt % and indeed may be less than 15 wt % based on the total weight of molecular sieves in the catalyst composition.

According to a further aspect of the present invention there is provided a process for the preparation of an oxygenate conversion catalyst, the process comprising preparing oxygenate conversion catalyst particles comprising a first molecular sieve having one-dimensional 10-membered ring channels, and a second molecular sieve having more-dimensional channels such that the resulting individual catalyst particles comprise both the first molecular sieve and second molecular sieve.

To form a catalyst the first and second molecular sieves are typically embedded in a matrix. For the purposes of this invention ‘matrix’ is herein referred to as including any filler and/or binder components.

For certain embodiments, a mixture comprising the first and second molecular sieves and matrix are spray dried to form the catalyst particles. Typically a mixture comprising the first and second molecular sieves are milled, either separately but preferably together, before the matrix is added to form a slurry that is spray dried.

Alternatively the first and second molecular sieves are co-crystallised or intergrown. For such embodiments a matrix is typically added after co-crystallisation and the resulting mixture is then spray dried. Co-crystallisation and intergrowth of two or more molecular sieves are well known processes to the skilled person and does not need any further explanation.

Preferably the catalyst particles prepared in accordance with the further aspect of the present invention are used in a process in accordance with the second aspect of the present invention.

The process of the invention allows maximising of olefin production, in particular ethylene and/or propylene production, more in particular a high ethylene make, from an oxygenate feedstock comprising e.g. methanol and/or dimethylether. It has been found that an oxygenate conversion catalyst according to the present invention is particularly effective for this purpose. It has been found particularly advantageous to apply this catalyst for converting a reaction mixture comprising an olefinic co-feed in addition to the oxygenate, to an olefinic product comprising ethylene and/or propylene.

Examples of an oxygenate that can be used as feedstock in the present invention include alcohols, such as methanol, ethanol, isopropanol, ethylene glycol, propylene glycol; ketones, such as acetone and methylethylketone; aldehydes, such as formaldehyde, acetaldehyde and propionaldehyde; ethers, such as dimethylether, diethylether, methylethylether, tetrahydrofuran and dioxane; epoxides such as ethylene oxide and propylene oxide; and acids, such as acetic acid, propionic acid, formic acid and butyric acid. Further examples are dialkyl carbonates such as dimethyl carbonate or alkyl esters of carboxylic acids such as methyl formate. Of these examples, alcohols and ethers are preferred.

Examples of preferred oxygenates include alcohols, such as methanol, ethanol, isopropanol, ethylene glycol, propylene glycol; and dialkyl ethers, such as dimethylether, diethylether, methylethylether. Cyclic ethers such as tetrahydrofuran and dioxane, are also suitable.

The oxygenate used in the process according to the invention is preferably an oxygenate which comprises at least one oxygen-bonded alkyl group. The alkyl group preferably is a C1-C4 alkyl group, i.e. comprises 1 to 4 carbon atoms; more preferably the alkyl group comprises 1 or 2 carbon atoms and most preferably one carbon atom. The oxygenate can comprise one or more of such oxygen-bonded C1-C4 alkyl groups. Preferably, the oxygenate comprises one or two oxygen-bonded C1-C4 alkyl groups.

More preferably an oxygenate is used having at least one Cl or C2 alkyl group, still more preferably at least one C1 alkyl group.

Preferably the oxygenate is chosen from the group of alkanols and dialkyl ethers consisting of dimethylether, diethylether, methylethylether, methanol, ethanol and isopropanol, and mixtures thereof.

Most preferably the oxygenate is methanol or dimethylether, or a mixture thereof.

Preferably the oxygenate feedstock comprises at least 50 wt % of methanol and/or dimethylether, more preferably at least 80 wt %, most preferably at least 90 wt %.

The oxygenate feedstock can be obtained from a prereactor, which converts methanol at least partially into dimethylether. In this way, water may be removed by distillation and so less water is present in the process of converting oxygenate to olefins, which has advantages for the process design and lowers the severity of hydrothermal conditions the catalyst is exposed to.

The oxygenate feedstock can comprise an amount of water, preferably less than 10 wt %, more preferably less than 5 wt %, based on the total weight of oxygenate feedstock. Preferably the oxygenate feedstock contains essentially no hydrocarbons other than oxygenates, i.e. less than 5 wt %, preferably less than 1 wt %, based on the total weight of oxygenate feedstock.

In one embodiment, the oxygenate is obtained as a reaction product of synthesis gas. Synthesis gas can for example be generated from fossil fuels, such as from natural gas or oil, or from the gasification of coal. Suitable processes for this purpose are for example discussed in Industrial Organic Chemistry, Klaus Weissermehl and Hans-Jürgen Arpe, 3rd edition, Wiley, 1997, pages 13-28. This book also describes the manufacture of methanol from synthesis gas on pages 28-30.

In another embodiment the oxygenate is obtained from biomaterials, such as through fermentation. For example by a process as described in DE-A-10043644.

In a particular embodiment the oxygenate feedstock is reacted to produce the olefinic product in the presence of an olefinic co-feed. By an olefinic composition or stream, such as an olefinic product, product fraction, fraction, effluent, reaction product or the like is understood a composition or stream comprising one or more olefins, unless specifically indicated otherwise. Other species can be present as well. Apart from olefins, the olefinic co-feed may contain other hydrocarbon compounds, such as for example paraffinic compounds. Preferably the olefinic co-feed comprises an olefinic portion of more than 50 wt %, more preferably more than 60 wt %, for example more than 70 wt %, which olefinic portion consists of olefin(s). The olefinic co-feed can also consist essentially of olefin(s).

Any non-olefinic compounds in the olefinic co-feed are preferably paraffinic compounds. Such paraffinic compounds are preferably present in an amount in the range from 0 to 50 wt %, more preferably in the range from 0 to 40 wt %, still more preferably in the range from 0 to 30 wt %.

By an olefin is understood an organic compound containing at least two carbon atoms connected by a double bond. The olefin can be a mono-olefin, having one double bond, or a poly-olefin, having two or more double bonds. Preferably olefins present in the olefinic co-feed are mono-olefins. C4 olefins, also referred to as butenes (1-butene, 2-butene, iso-butene, and/or butadiene), in particular C4 mono-olefins, are preferred components in the olefinic co-feed.

Preferably, when an olefinic co-feed is used, it is at least partially obtained by a recycle stream formed by recycling a suitable fraction of the reaction product comprising C4 olefin. The skilled artisan knows how to obtain such a fraction from the olefinic reaction product such as by distillation.

In one embodiment at least 70 wt % of the olefinic co-feed, during normal operation, is formed by the recycle stream, preferably at least 90 wt %, more preferably at least 99 wt %. Most preferably the olefinic co-feed is during normal operation formed by the recycle stream, so that the process converts oxygenate feedstock to predominantly light olefins without the need for an external olefins stream. During normal operation means for example in the course of a continuous operation of the process, for at least 70% of the time on stream. The olefinic co-feed may need to be obtained from an external source, such as from a catalytic cracking unit or from a naphtha cracker, during start-up of the process, when the reaction product comprises no or insufficient C4+ olefins.

The C4 fraction contains C4 olefin(s), but can also contain a significant amount of other C4 hydrocarbon species, in particular C4 paraffins, because it is difficult to economically separate C4 olefins and paraffins, such as by distillation.

In one embodiment the olefinic co-feed and preferably also the recycle stream comprises C4 olefins and less than 10 wt % of C5+ hydrocarbon species, more preferably at least 50 wt % of C4 olefins, and at least a total of 70 wt % of C4 hydrocarbon species.

The olefinic co-feed and preferably also the recycle stream, can in particular contain at least a total of 90 wt % of C4 hydrocarbon species. In one embodiment, the olefinic co-feed comprises less than 5 wt % of C5+ olefins, preferably less than 2 wt % of C5+ olefins, even more preferably less than 1 wt % of C5+ olefins, and likewise the recycle stream. In another embodiment, the olefinic co-feed, comprises less than 5 wt % of C5+ hydrocarbon species, preferably less than 2 wt % of C5+ hydrocarbon species even more preferably less than 1 wt % of C5+ hydrocarbon species, and likewise the recycle stream.

Thus in certain preferred embodiments, the olefinic portion of the olefinic co-feed, and of the recycle stream, comprises at least 90 wt % of C4 olefins, more preferably at least 99 wt %. Butenes as co-feed have been found to be particularly beneficial for high ethylene selectivity. Therefore one particularly suitable recycle stream consists essentially, i.e. for at least 99 wt %, of 1-butene, 2-butene (cis and trans), isobutene, n-butane, isobutene, butadiene.

In further embodiments the recycle stream can contain a larger fraction of C5 and/or higher olefins. It is for example possible to recycle more than 50% or substantially all of the C5 olefins in the reactor effluent.

In certain embodiments, the recycle stream can also comprise propylene. This may be preferred when a particularly high production of ethylene is desired, so that part or all of the propylene produced, such as at least 5 wt % thereof, is recycled together with C4 olefins.

The preferred molar ratio of oxygenate in the oxygenate feedstock to olefin in the olefinic co-feed depends on the specific oxygenate used and the number of reactive oxygen-bonded alkyl groups therein. Preferably the molar ratio of oxygenate to olefin in the total feed lies in the range of 20:1 to 1:10, more preferably in the range of 15:1 to 1:5.

In a preferred embodiment wherein the oxygenate comprises only one oxygen-bonded methyl group, such as methanol, the molar ratio preferably lies in the range of from 20:1 to 1:5 and more preferably in the range of 15:1 to 1:2.5.

In another preferred embodiment wherein the oxygenate comprises two oxygen-bonded methyl groups, such as for example dimethylether, the molar ratio preferably lies in the range from 10:1 to 1:10.

The expression ‘molecular sieve’ is used in the description and claims for a material containing small regular pores and/or channels and exhibiting catalytic activity in the conversion of oxygenate to olefin. The first molecular sieve having one-dimensional 10-membered ring channels and/or the second molecular sieve having more-dimensional channels (“more-dimensional molecular sieve”) can in particular be a zeolite or zeolites. A zeolite is understood to be an aluminosilicate molecular sieve, also referred to as aluminosilicate. Where reference is made in the description and in the claims to a molecular sieve, this can in particular be a zeolite. The first molecular sieve having one-dimensional 10-membered ring channels and/or the second molecular sieve having more-dimensional channels can be a mixture of different types of molecular sieves having the respective channel structures. So, for example, a mixture of ZSM-22 and ZSM-23 zeolites, both having one-dimensional 10-membered ring channels, can be used as first molecular sieve. Similarly, different more-dimensional molecular sieves can be mixed to form the second molecular sieve.

The process to prepare an olefin is carried out in presence of the first molecular sieve having one-dimensional 10-membered ring channels. These are understood to be molecular sieves having only 10-membered ring channels in one direction, which are not intersected by other channels, in particular other 8, 10 or 12-membered ring channels, from another direction.

Preferably, the first molecular sieve is a zeolite especially one selected from the group of TON-type (for example zeolite ZSM-22), MTT-type (for example zeolite ZSM-23), STF-type (for example SSZ-35), SFF-type (for example SSZ-44), EUO-type (for example ZSM-50), and EU-2-type molecular sieves or mixtures thereof.

MTT-type catalysts are more particularly described in e.g. U.S. Pat. No. 4,076,842. For purposes of the present invention, MTT is considered to include its isotypes, e.g., ZSM-23, EU-13, ISI-4 and KZ-1.

TON-type molecular sieves are more particularly described in e.g. U.S. Pat. No. 4,556,477. For purposes of the present invention, TON is considered to include its isotypes, e.g., ZSM-22, Theta-1, ISI-1, KZ-2 and NU-10.

EU-2-type molecular sieves are more particularly described in e.g. U.S. Pat. No. 4,397,827. For purposes of the present invention, EU-2 is considered to include its isotypes, e.g., ZSM-48.

In a further preferred embodiment a first molecular sieve of the MTT-type, such as ZSM-23, and/or a TON-type, such as ZSM-22 is used.

Molecular sieve and zeolite types are for example defined in Ch. Baerlocher and L. B. McCusker, Database of Zeolite Structures: http://www.izastructure.org/databases/, which database was designed and implemented on behalf of the Structure Commission of the International Zeolite Association (IZA-SC), and based on the data of the 4th edition of the Atlas of Zeolite Structure Types (W. M. Meier, D. H. Olson and Ch. Baerlocher).

A molecular sieve having more-dimensional channels is understood to have intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels.

The second molecular sieve comprises MEL-type aluminosilicate. MEL type aluminosilicate has a three-dimensional structure of intersecting 10-membered ring channels. The second molecular sieve can in particular be formed by only MEL-type aluminosilicate.

It is however possible that the second molecular sieve also comprises other more-dimensional molecular sieves.

The second molecular sieve can for example also include a FER type zeolite, which is a two-dimensional structure and has 8- and 10-membered rings intersecting each other. Preferably however the intersecting channels in the second molecular sieve are each 10-membered ring channels. Thus the second molecular sieve may be further comprise a zeolite, or a SAPO-type (silicoaluminophosphate) molecular sieve. More preferably however the second molecular sieve is a zeolite or aluminosilicate. The second molecular sieve can also comprise an MFI-type zeolite, in particular zeolite ZSM-5.

The presence of the second molecular sieve in the oxygenate conversion catalyst was found to improve stability (slower deactivation during extended runs) and hydrothermal stability compared to a catalyst with only the one-dimensional molecular sieve and without the more-dimensional molecular sieve. Without wishing to be bound by a particular hypothesis or theory, it is presently believed that this is due to the possibility for converting larger molecules by the second molecular sieve having more-dimensional channels, that were produced by the first molecular sieve having one-dimensional 10-membered ring channels, and which would otherwise form coke. Moreover an intimate mix of the first with the second molecular sieve, such that both are present in the individual catalyst particles, improves the selectivity towards ethylene and propylene, more in particular towards ethylene.

The weight ratio between the first molecular sieve having one-dimensional 10-membered ring channels, and the second molecular sieve having more-dimensional channels can be in the range of from 1:100 to 100:1. Preferably, the first molecular sieve is present in a weight majority. The weight ratio of first to second molecular sieve can be of from 1:1 to 100:1, more preferably in the range of from 9:1 to 2:1.

In special embodiments the oxygenate conversion catalyst can comprise less than 35 wt % of the second molecular sieve, based on the total molecular sieve in the oxygenate conversion catalyst, in particular less than 20 wt %, more in particular less than 18 wt %, still more in particular less than 15 wt %. Suitably at least 1 wt % of the second molecular sieve, based on the total molecular sieve, is present in the catalyst.

In one embodiment the oxygenate conversion catalyst can comprise more than 50 wt %, preferably at least 65 wt %, based on total molecular sieve in the oxygenate conversion catalyst, of the molecular sieve having one-dimensional 10-membered ring channels. The presence of a majority of such molecular sieve strongly determines the predominant reaction pathway.

Without wishing to be bound by a particular hypothesis or theory, it is currently believed that the reaction is dominated by a majority portion of the molecular sieve having one-dimensional 10-membered ring channels. In such molecular sieve an alcohol or ether oxygenate can be converted to an olefinic product by an initial alkylation step with an olefin from the olefinic co-feed, followed by cracking. The presence of a minority portion of a more-dimensional molecular sieve in the oxygenate conversion catalyst was found sufficient to significantly improve stability and hydrothermal stability compared to a catalyst with only the one-dimensional molecular sieve and without the more-dimensional molecular sieve.

In one embodiment, the first and second molecular sieves are used in their hydrogen form in the oxygenate conversion catalyst, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. When the molecular sieves are prepared in the presence of organic cations the molecular sieve may be activated by heating in an inert or oxidative atmosphere to remove organic cations, for example, by heating at a temperature over 500° C. for 1 hour or more. The sieves are typically obtained in the sodium or potassium form. The hydrogen form can then be obtained by an ion exchange procedure with ammonium salts followed by another heat treatment, for example in an inert or oxidative atmosphere at a temperature over 300° C. The molecular sieves obtained after ion-exchange are also referred to as being in the ammonium form.

In a preferred embodiment the first molecular sieve having one-dimensional 10-membered ring channels comprises at least one of a molecular sieve of the MTT-type and/or of the TON-type. Examples are ZSM-23 for MTT, and ZSM-22 for TON.

Suitably the molecular sieve having one-dimensional 10-membered ring channels has a Silica-to-Alumina ratio (SAR) in the range from 1 to 500. A particularly suitable SAR is less than 200, in particular 150 or less. A preferred range is from 10 to 200 or from 10-150. The SAR is defined as the molar ratio of SiO₂/Al₂O₃ corresponding to the composition of the molecular sieve.

For ZSM-22, a SAR in the range of 40-150 is preferred, in particular in the range of 70-120. Good performance in terms of activity and selectivity has been observed with a SAR of about 100.

For ZSM-23, a SAR in the range of 20-120 is preferred, in particular in the range of 30-80. Good performance in terms of activity and selectivity has been observed with a SAR of about 50.

Preferably the second molecular sieve having more-dimensional channels has a silica-to-alumina ratio (SAR) in the range from 1 to 1000. A SAR of 60 or higher is preferred, in particular 80 or higher, more preferably 100 or higher, still more preferably 150 or higher, such as 200 or higher. At higher SAR the percentage of C4 saturates in the C4 totals produced is minimized.

Catalyst particles are typically formulated from the molecular sieves, such as in a mixture or in combination within a matrix component such as a so-called binder material and/or a filler material. Other components can also be present in the formulation.

It is desirable to provide a catalyst having good mechanical or crush strength, because in an industrial environment the catalyst is often subjected to rough handling, which tends to break down the catalyst into powder-like material. The latter causes problems in the processing. Preferably the molecular sieve is therefore incorporated within a matrix such as a binder material. Examples of suitable materials in a formulation include active and inactive materials and synthetic or naturally occurring zeolites as well as inorganic materials such as clays, silica, alumina, silica-alumina, titania, zirconia and aluminosilicate or mixtures thereof. For present purposes, inert materials, such as silica, are preferred because they may prevent unwanted side reactions which may take place in case a more acidic material, such as alumina or silica-alumina is used.

Preferably the matrix and therefore the formulated catalyst (such as a combination of the first and second molecular sieves and the matrix) comprise non-zeolitic components. Typically at least 5 wt % of the formulated catalyst comprises non-zeolitic components, preferably at least 20 wt %.

Preferably the total molecular sieve content, especially where the molecular sieves are both zeolites, is at most 60 wt % of the formulated catalyst particles, especially at most 50 wt %; typically at least 10 wt %.

Silica binder is especially preferred where the molecular sieve comprises ZSM-22.

The oxygenate conversion catalyst can be further treated for improved performance, such as to further decrease the amount of by-products like aromatics and saturates in the oxygenate-to-olefins reaction. Further treatment e.g. be a treatment with a phosphorus containing compound such as phosphoric acid, a sulphur containing compound such as sulphuric acid or Group II metal species such as a calcium species; e.g. by impregnation and subsequent calcinations of the formulated catalyst so as to deposit a phosphorus, sulphur and/or Group II metal species on the catalyst. Also a treatment with a chelating agent or acid such as oxalic acid is possible.

Now the aspect of the present invention relating to the process for the preparation of an olefinic product will be discussed in more detail, which process comprises reacting an oxygenate feedstock in a reaction zone in the presence of oxygenate conversion catalyst according to the invention.

The process for the preparation of an olefinic product of the present invention can be carried out in a batch, continuous, semi-batch or semi-continuous manner. Preferably the process of the present invention is carried out in a continuous manner.

Since a second molecular sieve having more-dimensional channels is present in the oxygenate conversion catalyst particles, start up is possible without an olefinic co-feed from an external source. Such a molecular sieve is able to convert an oxygenate to an olefin-containing product, so that a recycle can be established. Molecular sieves with one-dimensional 10-membered ring channels such as ZSM-22 or ZSM-23 are typically not able to convert an oxygenate feed to an olefinic product stream with any useful conversion, unless an olefinic co-feed is provided.

In a particular embodiment an olefinic co-feed obtained from an external source may be used at start-up, and/or after start-up. Such olefins may for example be obtained from a steam cracker, a catalytic cracker, alkane dehydrogenation (e.g. propane or butane dehydrogenation). Further, such olefins can be bought from the market. In a special embodiment the olefins for such start-up are obtained from a previous process that converted oxygenates, with or without olefinic co-feed, to olefins. Such a previous process may have been located at a different location or it may have been carried out at an earlier point in time.

Typically the oxygenate conversion catalyst deactivates in the course of the process. Conventional catalyst regeneration techniques can be employed. The catalyst particles used in the process of the present invention can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. If desired, spent oxygenate conversion catalyst can be regenerated and recycled to the process of the invention. Spray-dried particles allowing use in a fluidized bed or riser reactor system are preferred.

Spherical particles are normally obtained by spray drying. Preferably the average particle size is in the range of 1-200 μm, preferably 50-100 μm.

The reactor system used to produce the olefins may be any reactor known to the skilled person and may for example contain a fixed bed, moving bed, fluidized bed, riser reactor and the like. In one embodiment a riser reactor system can be used, in particular a riser reactor system comprising a plurality of serially arranged riser reactors. In another embodiment, a fast fluidized bed reactor can be used.

In processes where a riser reactor system is preferred, a catalyst is required which has high attrition resistance to limit the catalyst losses by attrition. Such catalyst is typically formed of spray-dried catalyst particles. The composition of the catalyst particles strongly influence their resistance to attrition.

The reaction to convert oxygenates and optionally the olefinic co-feed to an olefinic product can be carried out over a wide range of temperatures and pressures. Suitably, however, the oxygenate feed and optional olefinic co-feed are contacted with the molecular sieve at a temperature in the range from 200° C. to 650° C. In a further preferred embodiment the temperature is in the range from 250° C. to 630° C., more preferably in the range from 300° C. to 620° C., most preferably in the range from 450° C. to 600° C. Preferably the reaction to produce the olefins is conducted at a temperature of more than 450° C., preferably at a temperature of 460° C. or higher, more preferably at a temperature of 490° C. or higher. At higher temperatures a higher activity and ethylene selectivity is observed. Molecular sieves having one-dimensional 10-membered ring channels can be operated under oxygenate conversion conditions at such high temperatures with acceptable deactivation due to coking, contrary to molecular sieves with smaller pores or channels, such as 8-membered ring channels. Temperatures referred to hereinabove represent reaction temperatures, and it will be understood that a reaction temperature can be an average of temperatures of various feed streams and the catalyst in the reaction zone.

In addition to the oxygenate, and the olefinic co-feed (when present), a diluent may be fed into the reactor system, for example in the range of from 0.01 to 10 kg diluent per kg oxygenate feed, in particular from 0.5 to 5 kg/kg. Any diluent known by the skilled person to be suitable for such purpose can be used. Such diluent can for example be a paraffinic compound or mixture of compounds. Preferably, however, the diluent is an inert gas. The diluent can be argon, nitrogen, and/or steam. Of these, steam is the most preferred diluent. It can be preferred to operate with a minimum amount of diluent, such as less than 500 wt % of diluent based on the total amount of oxygenate feed, in particular less than 200 wt %, more in particular less than 100 wt %. Operation without a diluent is also possible.

The olefinic reaction product is typically fractionated. The skilled artisan knows how to separate a mixture of hydrocarbons into various fractions, and how to work up fractions further for desired properties and composition for further use. The separations can be carried out by any method known to the skilled person in the art to be suitable for this purpose, for example by vapour-liquid separation (e.g. flashing), distillation, extraction, membrane separation or a combination of such methods. Preferably the separations are carried out by means of distillation. It is within the skill of the artisan to determine the correct conditions in a fractionation column to arrive at such a separation. He may choose the correct conditions based on, inter alia, fractionation temperature, pressure, trays, reflux and reboiler ratios.

At least a light olefinic fraction comprising ethylene and/or propylene and a heavier olefinic fraction comprising C4 olefins are normally obtained. In one embodiment the heavier olefinic fraction contains less than 10 wt % of C5+ hydrocarbon species. Preferably also a water-rich fraction is obtained. Also a lighter fraction comprising methane, carbon monoxide, and/or carbon dioxide can be obtained, as well as one or more heavy fractions comprising C5+ hydrocarbons. Such a heavy fraction, that is not being recycled, can for example be used as gasoline blending component.

In a particular aspect the present invention provides a process for the preparation of an olefinic product, which process comprises the step a) of reacting an oxygenate feedstock and an olefinic co-feed in a reactor in the presence of oxygenate conversion catalyst comprising both a first molecular sieve having one-dimensional 10-membered ring channels, and a second molecular sieve having more-dimensional channels, wherein the second molecular sieve comprises MEL-type aluminosilicate, to prepare an olefinic reaction effluent. Preferably the weight ratio between the one-dimensional molecular sieve and the further molecular sieve is in the range of from 1:1 to 100:1. In a preferred embodiment, this process comprises the further steps of b) separating the olefinic reaction effluent into at least a first olefinic fraction and a second olefinic fraction; and c) recycling at least part of the second olefinic fraction obtained in step b) to step a) as olefinic co-feed; and d) recovering at least part of the first olefinic fraction obtained in step b) as olefinic product.

In step b) of this process according to the invention the olefinic reaction effluent of step a) is separated (fractionated). At least a first olefinic fraction and a second olefinic fraction, preferably containing C₄ olefins, are obtained. The first olefinic fraction typically is a light olefinic fraction comprising ethylene, and the second olefinic fraction is typically a heavier olefinic fraction comprising C4 olefins.

Preferably also a water-rich fraction is obtained. Also a lighter fraction comprising contaminants such as methane, carbon monoxide, and/or carbon dioxide can be obtained and withdrawn from the process, as well as one or more heavy fractions comprising C5+ hydrocarbons, including C5+ olefins. Such heavy fraction can for example be used as gasoline blending component. For example, the first olefinic fraction can comprise at least 50 wt %, preferably at least 80 wt %, of C1-C3 species, the recycled part of the second olefinic fraction can comprise at least 50 wt % of C₄ species, a heavier carbonaceous fraction that is withdrawn from the process can comprise at least 50 wt % of C₅₊ species.

In step c) at least part of the second olefinic fraction, preferably containing C₄ olefins, obtained in step b) is recycled to step a) as olefinic co-feed.

Only part of the second olefinic fraction or the complete second olefinic fraction may be recycled to step a).

In the process also a significant amount of propylene is normally produced. The propylene can form part of the light olefinic fraction comprising ethene, and which can suitably be further fractionated into various product components. Propylene can also form part of the heavier olefinic fraction comprising C4 olefins.

The various fractions and streams referred to herein, in particular the recycle stream, can be obtained by fractionating in various stages, and also by blending streams obtained during the fractionation. Typically, an ethylene- and a propylene-rich stream of predetermined purity such as export quality will be obtained from the process, e.g. from a C2 or C3 splitter, and also a stream rich in C4 comprising C4 olefins and optionally C4 paraffins, such as an overhead stream from a debutaniser column receiving the bottom stream from a depropanizer column at their inlet. It shall be clear that the heavier olefinic fraction comprising C4 olefins, forming the recycle stream, can be composed from quantities of various fractionation streams. So, for example, some amount of a propylene-rich stream can be blended into a C4 olefin-rich stream. In a particular embodiment at least 90 wt % of the heavier olefinic fraction comprising C4 olefins can be the formed by the overhead stream from a debutaniser column receiving the bottom stream from a depropanizer column at their inlet, more in particular at least 99 wt % or substantially all.

Suitably the olefinic reaction product comprises less than 10 wt %, preferably less than 5 wt %, more preferably less than 2 wt %, of C6-C8 aromatics, e.g. less than 1 wt %, of C6-C8 aromatics, based on total hydrocarbon. Producing low amounts of aromatics is desired since any production of aromatics consumes oxygenate which is therefore not converted to lower olefins.

The present invention will now be discussed in more detail and by way of example at the hand of several embodiments.

### EXAMPLE 1

In this example according to the invention, dimethyl ether (DME) and 1-butene were reacted over an oxygenate conversion catalyst formed by MTT-type zeolite (ZSM-23 with a silica-to-alumina ratio (SAR) of 46) as first molecular sieve, mixed with MEL-type aluminosilicate (zeolite ZSM-11 with a SAR of 235) as second molecular sieve. Catalyst particles were obtained by mixing the individual zeolite powders, pressing the mix into tablets, breaking the tablets into pieces and sieved. The weight ratio between MTT and MEL mixtures in this example was 80/20 wt/wt, respectively. For catalytic testing, the sieve fraction of 40-60 mesh has been used. Prior to reaction, the fresh catalyst in its ammonium-form was treated ex-situ in air at 600° C. for 2 hours.

The reaction was performed using a quartz reactor tube of 3.6 mm internal diameter. The catalyst was heated in argon to the reaction temperature of 525° C. and a mixture consisting of 3 vol % dimethyl ether, 3 vol % 1-butene, 2 vol % steam balanced in argon was passed over the catalyst at atmospheric pressure (1 bar). Gas hourly space velocity was 15000 ml/(g_{cat}·h), based on total gas flow and the mass of zeolite catalyst g_{cat}. Periodically, the effluent from the reactor was analyzed by gas chromatography (GC) to determine the product composition. The composition has been calculated on a weight basis of all hydrocarbons analyzed.

Table 1 shows the resulting product composition for the zeolite catalyst for various times on stream. Cn refers to hydrocarbon species having n carbon atoms, Cn+ refers to hydrocarbon species having n or more carbon atoms (n being an integer) figures include all; Cn= refers to olefinic hydrocarbon species having n carbon atoms. The index sats refers to saturated carbon species, and tot or totals refer to all respective hydrocarbon species.

TABLE 1

| Oxygenate conversion | MTT SAR 46/MEL SAR 235 | | |
|---|---|---|---|
| catalyst | Weight ratio 80:20 | | |
| Time on stream (h) | 2 | 15 | 23 |
| DME conversion (%) | 100 | 100 | 100 |
| Methane (wt %) | 0.4 | 0.4 | 0.5 |
| Ethylene(wt %) | 19.5 | 13.5 | 11.3 |
| Propylene (wt %) | 46.6 | 44.4 | 43.3 |
| C4 totals (wt %) | 25.2 | 28.5 | 28.9 |
| C5 totals (wt %) | 3.3 | 7.7 | 10.6 |
| C6-C9 totals (wt %) | 3.5 | 4.1 | 4.1 |
| C6-C8 aromatics | 1.3 | 1.4 | 1.4 |
| (wt %) | | | |
| % C4 saturates of C4 | 3.7 | 4.3 | 4.3 |
| totals | | | |
| % C5 saturates of C5 | 19.7 | 14.6 | 11.4 |
| totals | | | |
| Ethylene/propylene | 0.42 | 0.3 | 0.26 |
| ratio (wt/wt) | | | |

The oxygenate conversion was in all cases excellent. Deactivation of the catalyst is observed by changing product composition over extended times on stream. Generally, the ethylene selectivity and the ethylene/propylene ratio decrease with increasing time on stream. C5 and higher hydrocarbons increase with increasing time on stream.

Although these experiments did not include a recycle of a product fraction, such recycle was simulated by feeding butene together with the oxygenate. In applying the process of the invention, a low concentration of paraffins in the reaction effluent, in particular of butane and pentane, more in particular of butane, is preferred. This is because it is difficult to economically separate olefins and paraffins with the same number of carbon atoms, in particular butene and butane, such as by distillation. In a preferred embodiment of the present invention, a butene fraction of the reaction effluent is recycled, and this fraction can contain a large portion or substantially all butane of the reaction effluent. Paraffins, in particular butane, can be regarded as inerts at typical oxygenation conditions over the zeolite catalysts, therefore a certain level of paraffins (butane) will build up. This level is the lower, the lower the concentration of paraffins (butane) in the reaction effluent is.

### COMPARATIVE EXAMPLE 2

Under the same conditions as discussed for Experiment 1, experiments were conducted for an oxygenate conversion catalyst consisting only of MTT zeolite (ZSM-23) with SAR=46, not according to the invention.

The results for various times on stream are shown in Table 2.

TABLE 2

| Oxygenate conversion | | | |
|---|---|---|---|
| catalyst | MTT SAR 46 | | |
| Time on stream (h) | 2 | 15 | 23 |
| DME conversion (%) | 100 | 100 | 100 |
| Methane (wt %) | 0.4 | 0.4 | 0.3 |
| Ethylene (wt %) | 19.8 | 11.3 | 6.9 |
| Propylene (wt %) | 48.9 | 45.3 | 39.3 |
| C4 totals (wt %) | 22. | 21.2 | 20.8 |
| C5 totals (wt %) | 3 | 16.1 | 26.8 |
| C6-C9 totals (wt %) | 4.6 | 4.8 | 5.4 |
| C6-C8 aromatics | 0.6 | 0.8 | 0.2 |
| (wt %) | | | |
| % C4 saturates of C4 | 2.3 | 2.5 | 2.6 |
| totals | | | |
| % C5 saturates of C5 | 11.7 | 4.4 | 2.8 |
| totals | | | |
| Ethylene/propylene | 0.41 | 0.25 | 0.18 |
| ratio (wt/wt) | | | |

The deactivation of zeolite ZSM-23 alone is much faster, as can be seen from the decreased ethylene make and the increased C5 and higher hydrocarbons. The ethylene yield is initially comparable to that in Example 1, but decreases with increasing time on stream.

### COMPARATIVE EXAMPLE 3

Under the same conditions as discussed for Experiment 1, experiments were conducted for two catalysts consisting of an MTT zeolite (ZSM-23) with SAR=46 mixed with an MFI-type zeolite with a SAR of 55, or 280, respectively, both catalysts not according to the invention. Catalyst particles were obtained by mixing the individual zeolite powders, pressing the mix into tablets, breaking the tablets into pieces and sieved. The weight ratio between MTT and MFI mixtures was 80/20 wt/wt in each case, respectively. For catalytic testing, the sieve fractions of 40-60 mesh have been used. Prior to reaction, the fresh catalyst in its ammonium-form was treated ex-situ in air at 600° C. for 2 hours. The results for various times on stream are shown in Tables 3 and 4.

TABLE 3

| Oxygenate conversion | MTT SAR 46/MFI SAR 55 | | |
|---|---|---|---|
| catalyst | Weight ratio 80:20 | | |
| Time on stream (h) | 2 | 15 | 23 |
| DME conversion (%) | 100 | 100 | 100 |
| Methane (wt %) | 1.1 | 1.6 | 1.7 |
| Ethylene (wt %) | 24.5 | 15.6 | 12.3 |
| Propylene (wt %) | 36.5 | 39.4 | 38.5 |
| C4 totals (wt %) | 17.8 | 20.4 | 22.7 |
| C5 totals (wt %) | 5.8 | 8.3 | 9.7 |
| C6-C9 totals (wt %) | 5 | 6 | 6.9 |
| C6-C8 aromatics | 9.3 | 8.5 | 8.2 |
| (wt %) | | | |
| % C4 saturates of C4 | 39.1 | 27 | 23.3 |
| totals | | | |
| % C5 saturates of C5 | 71.2 | 79.3 | 76.5 |
| totals | | | |
| Ethylene/propylene | 0.67 | 0.4 | 0.32 |
| ratio (wt/wt) | | | |

TABLE 4

| Oxygenate conversion | MTT SAR 46/MFI SAR 280 | | |
|---|---|---|---|
| catalyst | Weight ratio 80:20 | | |
| Time on stream (h) | 2 | 15 | 23 |
| DME conversion (%) | 100 | 100 | 100 |
| Methane (wt %) | 0.5 | 0.4 | 0.4 |
| Ethylene (wt %) | 17.4 | 12.3 | 10.6 |
| Propylene(wt %) | 45.2 | 42.5 | 41.1 |
| C4 totals (wt %) | 26.8 | 30.2 | 30.4 |
| C5 totals (wt %) | 3.8 | 8 | 10.5 |
| C6-C9 totals (wt %) | 3.9 | 4.6 | 4.9 |
| C6-C8 aromatics | 2.0 | 1.9 | 2 |
| (wt %) | | | |
| % C4 saturates of C4 | 4.9 | 6.4 | 6.8 |
| totals | | | |
| % C5 saturates of C5 | 23.9 | 21.4 | 19.2 |
| totals | | | |
| Ethylene/propylene | 0.38 | 0.29 | 0.26 |
| ratio (wt/wt) | | | |

With increasing SAR of the ZSM-5 component, a decreasing amount of C4 saturates in the total C4 portion of the reaction effluent is observed, which is preferred. Also with increasing SAR of the ZSM-5 component the amount of aromatics and of other C5+ hydrocarbons decreases, which is preferred for optimum conversion of oxygenate to lower olefins ethylene and propylene. Compared with Example 1, a significantly larger fraction of the product is aromatics and saturates, which are unwanted by-products. This is the case even when the SAR ratio of MFI is higher than that of MEL in Example 1 (Table 4). Moreover, the total make of lower olefins ethylene+propylene is higher in Example 1 for all times on stream.

### EXAMPLE 4

In this example according to the invention, dimethyl ether (DME) and 1-butene were reacted over an oxygenate conversion catalyst formed by a TON-type zeolite (ZSM-22 with a silica-to-alumina ratio (SAR) of 108) as first molecular sieve, mixed with a MEL-type aluminosilicate (zeolite ZSM-11 with a SAR of 235) as second molecular sieve. Catalyst particles were obtained by mixing the individual zeolite powders, pressing the mix into tablets, breaking the tablets into pieces and sieved. The weight ratio between TON and MEL mixtures in this example was 80/20 wt/wt, respectively. For catalytic testing, the sieve fraction of 40-60 mesh has been used. Prior to reaction, the fresh catalyst in its ammonium-form was treated ex-situ in air at 600° C. for 2 hours.

The reaction was performed using a quartz reactor tube of 3.6 mm internal diameter. The catalyst was heated in argon to the reaction temperature of 525° C. and a mixture consisting of 20 vol % dimethyl ether, 20 vol % 1-butene, 2 vol % steam balanced in argon was passed over the catalyst at atmospheric pressure (1 bar). Gas hourly space velocity was 15000 ml/(g_{cat}·h), based on total gas flow and the mass of zeolite catalyst g_{cat}. Periodically, the effluent from the reactor was analyzed by gas chromatography (GC) to determine the product composition. The composition has been calculated on a weight basis of all hydrocarbons analyzed.

Table 5 shows the resulting product composition for the zeolite catalyst for various times on stream.

TABLE 5

| Oxygenate conversion | TON SAR 108/MEL SAR 235 | | |
|---|---|---|---|
| catalyst | Weight ratio 80:20 | | |
| Time on stream (h) | 1 | 2.5 | 5.5 |
| DME conversion (%) | 100 | 100 | 100 |
| Methane (wt %) | 0.4 | 0.4 | 0.4 |
| Ethylene (wt %) | 14.7 | 10.3 | 7.1 |
| Propylene (wt %) | 39.8 | 38.9 | 37.3 |
| C4 totals (wt %) | 26.4 | 30.4 | 31.5 |
| C5 totals (wt %) | 7.7 | 11.1 | 15.8 |
| C6-C9 totals (wt %) | 9.4 | 7.2 | 6.4 |
| C6-C8 aromatics | 1.6 | 1.8 | 1.5 |
| (wt %) | | | |
| % C4 saturates of C4 | 8.3 | 6 | 4.7 |
| totals | | | |
| % C5 saturates of C5 | 12.2 | 10 | 6.7 |
| totals | | | |
| Ethylene/propylene | 0.37 | 0.27 | 0.19 |
| ratio (wt/wt) | | | |

The oxygenate conversion was in all cases excellent. Deactivation of the catalyst is observed by changing product composition over extended times on stream. Generally, the ethylene selectivity and the ethylene/propylene ratio decrease with increasing time on stream. C5 and higher hydrocarbons increase with increasing time on stream.

### COMPARATIVE EXAMPLE 5

Under the same conditions as discussed for Experiment 4, experiments were conducted for a zeolite catalyst consisting only of TON zeolite (ZSM-22) with SAR=108, not according to the invention.

The results for various times on stream are shown in Table 6.

TABLE 6

| Oxygenate conversion | | | |
|---|---|---|---|
| catalyst | TON SAR 108 | | |
| Time on stream (h) | 1 | 2.5 | 5.5 |
| DME conversion (%) | 100 | 100 | 99.7 |
| Methane (wt %) | 0.4 | 0.3 | 0.3 |
| Ethylene (wt %) | 14.7 | 9.1 | 1.9 |
| Propylene (wt %) | 41.6 | 40.6 | 20.7 |
| C4 totals (wt %) | 23.7 | 21.4 | 18.3 |
| C5 totals (wt %) | 9.6 | 21.6 | 42.1 |
| C6-C9 totals (wt %) | 8.1 | 6.1 | 16.5 |
| C6-C8 aromatics | 1.8 | 0.8 | 0.5 |
| (wt %) | | | |
| % C4 saturates of C4 | 3.9 | 2.5 | 4.9 |
| totals | | | |
| % C5 saturates of C5 | 2.9 | 1.8 | 0.8 |
| totals | | | |
| Ethylene/propylene | 0.35 | 0.22 | 0.09 |
| ratio (wt/wt) | | | |

The deactivation of zeolite ZSM-22 alone is much faster, as can be seen from the decreased ethylene make and the increased C5 and higher hydrocarbons. The ethylene yield is initially comparable to that in Examples 4, but decreases with increasing time on stream. The C5 and higher hydrocarbons are always higher at the same time on stream compared to Example 4.

### COMPARATIVE EXAMPLE 6

Under the same conditions as discussed for Experiment 4, experiments were conducted for a zeolite catalyst consisting a TON zeolite (ZSM-22) with SAR=108 mixed with an MFI-type zeolite with a SAR of 280, not according to the invention. Catalyst particles were obtained by mixing the individual zeolite powders, pressing the mix into tablets, breaking the tablets into pieces and sieved. The weight ratio between MTT and MFI mixtures in this example was 80/20 wt/wt, respectively. For catalytic testing, the sieve fraction of 40-60 mesh has been used. Prior to reaction, the fresh catalyst in its ammonium-form was treated ex-situ in air at 600° C. for 2 hours. The results for various times on stream are shown in Table 7.

TABLE 7

| Oxygenate conversion | TON SAR 108/MFI SAR 280 | | |
|---|---|---|---|
| catalyst | Weight ratio 80:20 | | |
| Time on stream (h) | 1 | 2.5 | 5.5 |
| DME conversion (%) | 100 | 100 | 100 |
| Methane (wt %) | 0.4 | 0.3 | 0.3 |
| Ethylene (wt %) | 15.3 | 10.3 | 6.7 |
| Propylene (wt %) | 39.4 | 37.7 | 35.3 |
| C4 totals (wt %) | 25.2 | 29.2 | 30.5 |
| C5 totals (wt %) | 7.8 | 11.4 | 16.4 |
| C6-C9 totals (wt %) | 8.6 | 7.9 | 7.9 |
| C6-C8 aromatics | 3.3 | 3.1 | 2.7 |
| (wt %) | | | |
| % C4 saturates of C4 | 10 | 9.8 | 8.9 |
| totals | | | |
| % C5 saturates of C5 | 15.2 | 21.3 | 16.1 |
| totals | | | |
| Ethylene/propylene | 0.39 | 0.27 | 0.19 |
| ratio (wt/wt) | | | |

With addition of ZSM-5 component, the aromatics and saturates make is higher than in Example 4, even though the SAR ratio of MFI is higher in Table 7 than that of MEL in Example 4.

1. An oxygenate conversion catalyst comprising both a first molecular sieve having one-dimensional 10-membered ring channels, and a second molecular sieve having more-dimensional channels, wherein the second molecular sieve comprises MEL-type aluminosilicate.

2. An oxygenate conversion catalyst according to claim 1, comprising catalyst particles, wherein individual catalyst particles comprise both the first molecular sieve and the second molecular sieve.

3. An oxygenate conversion catalyst according to claim 2, wherein an average distance between a crystal of the first molecular sieve and a nearest crystal of the second molecular sieve is less than an average particle size of the catalyst particles.

4. An oxygenate conversion catalyst according to claim 1 wherein the Silica-to-Alumina molar ratio of the first molecular sieve is less than 200.

5. An oxygenate conversion catalyst according to claim 1 wherein the molecular sieve having one-dimensional 10-membered ring channels comprises at least one of a molecular sieve of the MTT-type and the TON-type.

6. An oxygenate conversion catalyst according to claim 1 wherein the MEL-type aluminosilicate has a Silica-to-Alumina ratio (SAR) of at least 60.

7. An oxygenate conversion catalyst according to claim 1 wherein a bed of catalyst particles comprises at least 1 wt % and less than 50 wt % of the second molecular sieve, based on the total weight of first and second molecular sieves in the catalyst composition.

8. An oxygenate conversion catalyst according to claim 1, further comprising a matrix and a total of 60 wt % or less of the first and second molecular sieves, based on the total catalyst particles.

9. A process for the preparation of an olefinic product, which process comprising reacting an oxygenate feedstock in a reaction zone in the presence of an oxygenate conversion catalyst as claimed in claim 1, to prepare an olefinic reaction product.

10. A process according to claim 9, wherein the reaction of the oxygenate feedstock is performed in the presence of an olefinic co-feed.

11. A process for the preparation of an oxygenate conversion catalyst, the process comprising preparing oxygenate conversion catalyst particles comprising both a first molecular sieve having one-dimensional 10-membered ring channels, and a second molecular sieve having more-dimensional channels, wherein the second molecular sieve comprises MEL-type aluminosilicate.

12. A process according to claim 11, wherein the first and second molecular sieves are embedded in a matrix.

13. A process according to claim 12, wherein a slurry comprising the first and second molecular sieves and the matrix is spray dried to form the catalyst particles.

14. A process according to claim 11, wherein crystals of the first and second molecular sieves are grown together.
